(19) United States
(12) Patent Application Publication
(10) Pub. No.: US 2011/0015293 A1
(43) Pub. Date: Jan. 20, 2011

(54) POLYETHER NATURAL OIL POLYOLS AND POLYMERS THEREOF

(75) Inventors: Hongming Ma, Lake Jackson, TX (US); Jorge Jimenez, Lake Jackson, TX (US); Dwight D. Latham, Clute, TX (US); David A. Babb, Lake Jackson, TX (US); Imran Munshi, Au (CH); Christopher Noakes, Richterswil (CH)

Correspondence Address: The Dow Chemical Company, P.O. BOX 1967, 2040 Dow Center, Midland, MI, 48641, US

(73) Assignee: Dow Global Technologies Inc., Midland, MI (US)

(21) Appl. No.: 12/933,293

(22) PCT Filed: Mar. 20, 2009
(86) PCT No.: PCT/US09/37751

§ 371 Date: Sep. 17, 2010

Related U.S. Application Data
(60) Provisional application No. 61/144,540, filed on Jan. 14, 2009.
(60) Provisional application No. 61/038,274, filed on Mar. 20, 2008.

(51) Int. Cl. C08J 9/00; C07C 59/147; C08G 18/72

(52) U.S. Cl. 521/174; 554/120; 528/77

(57) ABSTRACT

A polyether NOP comprises at least two natural oil moieties separated by a molecular structure having at least about 19 ether groups or by a polyether molecular structure having an equivalent weight of at least about 480, said polyol referred to hereinafter as a polyether NOP. The polyether NOP is preferably prepared by a process comprising admixing at least one polyether initiator having an equivalent weight of at least about 480 or at least about 19 ether groups per active hydrogen group with at least one natural oil or derivative thereof under reaction conditions such that at least one polyether NOP is formed. The invention includes reaction products of the polyether NOP or combinations thereof, optionally and preferably in admixture with at least one other polyol which preferably includes at least one polyether polyol and at least one monomer reactive therewith, preferably at least one polyisocyanate to form a polyurethane, preferably in the form of a foam, especially a high resilience foam. The invention includes articles comprising at least one polyether NOP, at least one polymer thereof, or at least one foam thereof.

### CROSS-REFERENCE TO RELATED APPLICATIONS

This application claims benefit of U.S. Provisional Patent Application Ser. No. 61/038,274, Mar. 20, 2008, entitled “Polyether Natural Oil Polyols and Polymers Thereof” and of U.S. Provisional Patent Application Ser. No. 61/144540, Jan. 14, 2009, entitled “Development of Renuva(™) Based Polyols for High Resilience Foam Applications”, both of which are herein incorporated by reference.

### BACKGROUND

1. Field of Invention

This invention relates to polyols, especially polyols prepared using natural oils or derivatives thereof and to polymers prepared from such polyols, particularly polyurethane or urea polymers.

Polyols that are derived from natural oils or derivatives of natural oils (NOPs) have been useful in a number of areas including making polyurethanes. The polyurethanes are useful in areas including foams and coatings, such as disclosed for instance in such references as WO2004096883, WO2004096882, WO2006047698, WO2006047432, WO2006065345, WO2006047433, WO2006047431, WO2006047434, WO2006118995, WO2007019051, and WO2007019063. However, some properties of such polyols could be improved for certain applications. For instance, it would be desirable to achieve greater compatibility, miscibility or a combination thereof with conventional polyols used in polyurethane applications, especially with polyols containing ethylene oxide blocks, than is observed with currently available NOPs. This would be useful when combinations of conventional polyols and NOP's are desired, and especially when it would be desirable to store such mixtures. Similarly, when it is desirable to use more water than is conveniently miscible with a NOP, it would be desirable to increase water miscibility over that available with currently available NOP's. It would also be desirable to improve properties of polymers produced by the reaction of these polyols with such comonomers as isocyanates in at least one of tensile strength, elongation, resiliency or other physical properties important to the resulting polymer or its final form such as a foam. Furthermore, it would be desirable to improve processibility during the production of polyurethane polymers and their final forms, especially polyurethane foams, when NOPs are present in the formulation. Examples of processibility improvements are indicated by a broader tin latitude (defined as the range of tin catalyst concentration over which good quality foams can be made), better foam flowability, higher open cell content and in a molded application, a lower force to crush after the foam is made.

### SUMMARY OF THE INVENTION

It has now been found that NOP's or mixtures of NOP's wherein the natural oil moieties are separated by a molecular structure having ether groups as further described herein and an equivalent weight of at least about 480, as compared with an operably equivalent NOP having natural oil moieties separated by a molecular structure of lower equivalent weight or one not having ether groups, achieve at least one of (a) greater miscibility or compatibility with conventional polyether polyols, (b) greater miscibility or compatibility with water, (c) greater processibility in making a polyurethane, preferably a polyurethane foam, (d) increased tensile strength in a polyurethane; (e) increased elongation in a polyurethane, or (f) increased resiliency in a polyurethane, where, in each instance, the comparative polyurethanes are produced by the same method using the same formulation except that the polyether NOP of the invention are replaced by an operably comparable conventional NOP.

In one aspect, the invention is a polyol which comprises at least two natural oil moieties separated by a molecular structure having at least about 19 ether groups or separated by a polyether molecular structure having an equivalent weight of at least about 480.

The invention includes a method of making a polyether NOP comprising admixing at least one polyether initiator having an equivalent weight of at least about 480 or an average at least about 9.5 ether groups per active hydrogen group with at least one natural oil or derivative thereof under reaction conditions such that at least one polyether NOP is formed.

The invention additionally includes a polymer comprising the reaction product of a polyether NOP of the invention and at least one other monomer different from a polyether NOP and reactive therewith, which polymer is preferably a polyurethane. The invention additionally includes a method of making a polymer comprising admixing at least one polyether NOP of the invention with at least one monomer reactive therewith under reaction conditions such that a polymer is formed.

Furthermore, the invention includes articles comprising at least one polymer or polyether NOP of the invention as well as a composition comprising at least one polyether NOP of the invention. Such articles include high resiliency foams made from at least one polyether NOP of the invention.

### DETAILED DESCRIPTION OF THE INVENTION

Definitions

The term “miscibility” as used herein refers to the tendency or capacity of two or more liquids to form a uniform blend or mixture. Liquids are immiscible if separate liquid phases or domains are formed. It is possible for each of the separated phases to contain substantial amounts of each of the two or multiple components of the mixture. Separation may be visible as separate layers or as smaller domains of one liquid in another which may be visible as, for instance, haze. For the purposes of this invention, miscibility is determined by allowing a blend to sit without stirring for 2 weeks and observing the degree of haziness or formation of separate layers. Two or more liquids are also referred to herein as “compatible” if they are miscible.

The term “processibility” as used herein refers to a noticeable advantage during or soon after the production of a foam. Examples of processibility improvements are indicated by a broader tin latitude (defined as the range of tin catalyst concentration over which good quality foams can be made), better foam flowability, higher open cell content and, in a molded application, a lower force to crush after the foam is made.

The term “resilience” or “resiliency” is used to refer to the quality of a foam perceived as springiness. It is measured according to the procedures of ASTM D3574 Test H. This ball rebound test measures the height a dropped steel ball of known weight rebounds from the surface of the foam when dropped under specified conditions and expresses the result as a percentage of the original drop height. As measured according to the ASTM test, a high resilience (HR) foam exhibits a resiliency of at least about 40 percent, more preferably at least about 42 percent, most preferably at least about 48 percent and advantageously at least about 50 percent.

The term “density” is used herein to refer to weight per unit volume of a foam. In the case of polyurethane foams, the density is determined according to the procedures of ASTM D3574-01, Test A.

The term “air flow” refers to the volume of air which passes through a 1.0 inch (2.54 cm) thick 2 inch×2 inch (5.08 cm) square section of foam at 125 Pa (0.018 psi) of pressure. Units are expressed in cubic decimeters per second and converted to standard cubic feet per minute. A representative commercial unit for measuring air flow is manufactured by TexTest AG of Zurich, Switzerland and identified as TexTest Fx3300. This measurement follows ASTM D 3574 Test G.

The term “75% CS” stands for dry compression set test measured at the 75 percent compressive deformation level and parallel to the rise direction in the foam. This test is used herein to correlate in-service loss of cushion thickness and changes in foam thickness. The compression set is determined according to the procedures of ASTM D 3574-95, Test I. and is measured as percentage of original thickness of the sample. Similarly, “50% CS” refers to the same measurement (compression set), but this time measured at 50 percent compressive deformation level of the sample, parallel to the rise direction in the foam.

The term “25% IFD” refers to a measure of the indentation force deflection of a flexible material (for instance, foam) measured as the force in pounds force or N required to compress a 100 mm thick sample no smaller than 380 mm square, to 25 percent indentation after 2 precycles. The force is measured after 60 seconds. The IFD is measured according to the procedures of ASTM 3574 B1. Similarly, “65% IFD” refers to the same measurement to 65% indentation.

The term “tensile strength” as applied to a foam is used herein to refer to the maximum force which a dogbone shaped foam sample can bear while being extended under linear (uniaxial) extensional force. The stress is increased until the material reaches a break point at which time the load and extension at break are used to calculate the tensile strength and the elongation, all determined according to the procedures of ASTM D-3574, Test E and is measured in pounds per square inch (PSI) or kiloPascals (kPa).

The term “ultimate elongation” as applied to a polymer is used herein to refer to the linear extension which a sample of foam can attain before rupture. The foam is tested by the same method used to determine tensile strength, and the result is expressed as a percentage of the original length of the foam sample according to the procedures of ASTM D-3574, Test E.

The term “NCO Index” means isocyanate index, as that term is commonly used in the polyurethane art. As used herein as the equivalents of isocyanate, divided by the total equivalents of isocyanate-reactive hydrogen containing materials, multiplied by 100. Considered in another way, it is the ratio of isocyanate-groups over isocyanate-reactive hydrogen atoms present in a formulation, given as a percentage. Thus, the isocyanate index expresses the percentage of isocyanate actually used in a formulation with respect to the amount of isocyanate theoretically required for reacting with the amount of isocyanate-reactive hydrogen used in a formulation.

As used herein, “polyol” refers to an organic molecule having an average of greater than 1.0 hydroxyl groups per molecule. It may also include other functionalities, that is, other types of functional groups.

As used herein the term “conventional polyol” is any polyol within the skill in the art commonly used in the formation of polyurethane or urea polymers. Conventional polyether polyols are formed from at least one alkylene oxide, preferably ethylene oxide, propylene oxide or a combination thereof, and not having a part of the molecule derived from a vegetable or animal oil, a polyol of the type commonly used in making polyurethane foams, particularly for the practice of this invention, HR (high resilience) polyurethane foams. Polyether polyols are prepared by methods within the skill in the art such as by alkoxylation of suitable starter molecules. Such a method generally involves reacting an initiator such as, water, ethylene glycol, or propylene glycol, glycerol, sorbitol or blends thereof with an alkylene oxide in the presence of a catalyst. Ethylene oxide, propylene oxide, butylene oxide, or a combination of these oxides can be particularly useful for the alkoxylation reaction. A polyether polyol, for instance polyoxyethylene polyol can contain alkyl substituents. The process for producing polyether polyols can involve a heterogeneous feed of a mixture of alkylene oxides, a sequential feed of pure or nearly pure alkylene oxide polyols to produce a polyol with blocks of single components, or a polyol which is capped with, for example, ethylene oxide or propylene oxide. Other types of conventional polyols include polyester polyols, polybutadiene polyols, polyether siloxane polyols, poly tetrahydrofuran polyols and combinations thereof. These types of polyols are all known and used in polyurethane chemistry.

The term “natural oil polyol” (hereinafter NOP) is used herein to refer to compounds having hydroxyl groups which compounds are isolated from, derived from or manufactured from natural oils, including animal and vegetable oils, preferably vegetable oils. Examples of vegetable and animal oils that may be used include, but are not limited to, soybean oil, safflower oil, linseed oil, corn oil, sunflower oil, olive oil, canola oil, sesame oil, cottonseed oil, palm oil, rapeseed oil, tung oil, fish oil, or a blend of any of these oils. Alternatively, any partially hydrogenated or epoxidized natural oil or genetically modified natural oil can be used to obtain the desired hydroxyl content. Examples of such oils include, but are not limited to, high oleic safflower oil, high oleic soybean oil, high oleic peanut oil, high oleic sunflower oil (such as NuSun sunflower oil), high oleic canola oil, and high erucic rapeseed oil (such as Crumbe oil). Natural oil polyols are well within the knowledge of those skilled in the art, for instance as disclosed in Colvin et al., UTECH Asia, Low Cost Polyols from Natural Oils, Paper 36, 1995 and “Renewable raw materials—an important basis for urethane chemistry:” Urethane Technology: vol. 14, No. 2, April/May 1997, Crain Communications 1997, WO 01/04225, WO 2004/096882; WO 2004/096883; U.S. Pat. No. 6,686,435, U.S. Pat. No. 6,433,121, U.S. Pat. No. 4,508,853, U.S. Pat. No. 6,1074,03, US Pregrant publications 20060041157, and 20040242910. For purposes of this invention, petroleum oils are distinguished from natural oils.

The term “natural oil based polyol” is used herein to refer to NOP compounds which are derived from natural oils. For instance, natural oils or isolates therefrom are reacted with compounds ranging from air, carbon monoxide or oxygen to organic compounds including amines, aldehydes, carboxylic acids, and alcohols. Frequently, unsaturation in the natural oil is converted to hydroxyl groups or to a group which can subsequently be reacted with a compound that has hydroxyl groups such that a polyol is obtained. Such reactions are discussed in the references in the preceding paragraph.

The term “natural oil or derivative thereof” is used herein to refer to a natural oil as previously discussed or any substance, compound or combination thereof chemically or physically, preferably chemically, derivable from a natural oil. Multiple chemical reactions are optionally required.

The term “vegetable oil based (VOB) monomer” is used herein to refer to the monomers derived from fatty acids as described in WO2004096882, which is hereby incorporated herein by reference to the extent permitted by law and which also represents the knowledge of those skilled in the art. These VOB monomers are based on fatty acids and are preferably alkyl esters, more preferably methyl esters of the fatty acids, which have at least one hydroxyl group, more preferably resulting from reaction at sites of unsaturation, most preferably resulting from hydroformylation at sites of unsaturation. Most preferably, the VOB monomers are methyl esters of at least one fatty acid having at least one hydroxyl group, preferably at least one hydroxyl methyl group, more preferably having at least one hydroxymethyl group where the original fatty acid had a double carbon to carbon bond, most preferably having at least one hydroxymethyl group at each site of a double bond on the original fatty acid.

The term “natural oil moiety” is used herein to refer to a molecule or, preferably, a part of a molecule which is derived from or derivable from a natural oil. The moiety is optionally derivable from an entire oil molecule such as castor oil or from a portion of an oil molecule such as from a fatty acid or a fatty acid ester. It is optionally in the form obtained from the natural source, such as castor oil, or is the result of one or more chemical or physical processes such as the hydroxymethylated fatty acids or esters taught in such references as WO2004096882 and WO2004096883, the epoxidized fatty acids or triglycerides such as are taught in such references as US20060041157 or a combination of such molecules such as oligomerized hydroxymethyl fatty acids or their esters. The term, as used herein, is applied to collectively to contiguous parts of a molecule. That is, a series of molecular structures, such as an oligomer of fatty acids, is considered one “natural oil moiety.” Thus, as used in the description of the present invention, a structure which has only two oligomers of fatty acids separated by a polyether moiety has 2 fatty acid moieties, not as many fatty acid moieties as there are fatty acids in the chains.

The term “hydroxyl number” indicates the concentration of hydroxyl moieties in a composition of polymers, particularly polyols. A hydroxyl number represents mg KOH/g of polyol. A hydroxyl number is determined by acetylation with pyridine and acetic anhydride in which the result is obtained as the difference between two titrations with KOH solution. A hydroxyl number may thus be defined as the weight of KOH in milligrams that will neutralize the acetic anhydride capable of combining by acetylation with 1 gram of a polyol. A higher hydroxyl number indicates a higher concentration of hydroxyl moieties within a composition. A description of how to determine the hydroxyl number for a composition can be found in texts well-known in the art, for example in Woods, G., The ICI Polyurethanes Book—2nd ed. (ICI Polyurethanes, Netherlands, 1990).

The term “primary hydroxyl group” means a hydroxyl group (—OH) on a carbon atom which has only one other carbon atom attached to it, (preferably which has only hydrogen atoms attached thereto) (—CH₂—OH).

The term “ether group” is used herein to refer to any oxygen atom between two carbon atoms. The term “polyether” refers to a molecule having more than one ether group, preferably at least two ether groups separated by aliphatic (that is, by —CH₂—) or aromatic carbon atoms or combination thereof.

The term “functionality” particularly “polyol functionality” is used herein to refer to the number of hydroxyl groups in a polyol unless other functional groups are particularly identified. In the case of a mixture of compounds the functionality is the average functionality. It is obtained in the same manner as the average functionality number of the formyl or hydroxymethyl functionality explained hereinafter.

For the purposes of this invention, the term “average functionality number” (AFN) shall mean the average number of functional groups of a mixture of compounds having similar types of functional groups, for instance hydroxyl groups. In an illustration calculating the AFN of formyl or hydroxymethyl functionality per aldehyde or alcohol monomer composition, each sample of aldehyde or alcohol composition may be expressed as comprising the following components

A+B+C+D+E+F=1.0 (Eq. 1)

wherein A=mole fraction of saturates;
B=mole fraction of mono-aldehyde or mono-alcohol;
C=mole fraction of di-aldehyde or diol;
D=mole fraction of tri-aldehyde or triol;
E=mole fraction of lactols, lactones, and cyclic ethers;
F=mole fraction of dimers and heavies.

Based on the above composition, the average functionality number (AFN) can be calculated as follows:

AFN=0A+1B+2C+3D+1E+2F (Eq. 2)

wherein each mole fraction is multiplied by the number of formyl or hydroxymethyl functionalities per fatty acid chain of that fraction. More explicitly, the number of formyl or hydroxymethyl functionalities per fraction is as follows: (A) 0 for unsaturates, (B) 1 for mono-als and mono-ols, (C) 2 for dials and diols, (D) 3 for tri-als and triols. Fraction E comprising lactols, lactones, and cyclic ethers is taken to have a functionality of 1. Fraction F comprising dimers and heavies is taken to have a functionality of 2. Since the saturates (A) contribute no functionality, the first term of Equation 2 is zero; and equation (2) is reduced to the following:

AFN=1B+2C+3D+1E+2F (Eq. 3)

Often, the mole fractions of mono-, di-, and tri-substituted components (B, C, and D) can be based upon the molecular weight of the C18 component of the seed oil. Typically, the C16 and C20 components occur in small quantities that may, in fact, balance each other out in the calculation of mole fraction. Such a guideline should not, however, be taken as a requirement of this invention. In seed oils, wherein a C16, C20, or other carbon chain other than C18 occurs in significant quantity, it may be necessary to separate every component of the mono-, di-, and tri-substituted fractions and calculate their individual contributions to the mole fractions of B, C, and D.

The average functionality number of the alcohol composition may also be determined empirically by means of American Standard Test Method 4274 for determining hydroxyl number. Generally, the empirical method correlates closely with the calculated method. Such an empirical method is not at the present time available for determining the average functionality number of the aldehyde composition.

The term “active hydrogen functionality” is used herein to refer to the number of active hydrogen atoms, that is, hydrogen atoms available for reaction, more reactive than the hydrogen atoms on carbon in a polyol. A functional group having at least one active hydrogen atom is referred to as an active hydrogen group. Such groups are exemplified by hydroxyl groups, amine groups, sulfides and combinations thereof. The active hydrogen functionality of a mixture is the average active hydrogen functionality obtained in the same manner as was described for the average formyl or hydroxymethyl functionality

All percentages, preferred amounts or measurements, ranges and endpoints thereof herein are inclusive, that is, “less than about 10” includes about 10. “At least” is, thus, equivalent to “greater than or equal to,” and “at most” is, thus, equivalent “to less than or equal to.” Numbers herein have no more precision than stated. Thus, “115.” includes at least from 114.5 to 115.49. Furthermore, all lists are inclusive of combinations of two or more members of the list. All ranges from a parameter described as “at least,” “greater than,” “greater than or equal to” or similarly, to a parameter described as “at most,” “up to,” “less than,” “less than or equal to” or similarly are preferred ranges regardless of the relative degree of preference indicated for each parameter. Thus a range that has an advantageous lower limit combined with a most preferred upper limit is preferred for the practice of this invention. All amounts, ratios, proportions and other measurements are by weight unless stated otherwise. All percentages refer to weight percent based on total composition according to the practice of the invention unless stated otherwise. Except in the examples, or where otherwise indicated, all numbers expressing quantities, percentages, OH numbers, functionalities and so forth in the specification are to be understood as being modified in all instances by the term “about.” Unless stated otherwise or recognized by those skilled in the art as otherwise impossible, steps of processes described herein are optionally carried out in sequences different from the sequence in which the steps are discussed herein. Furthermore, steps optionally occur separately, simultaneously or with overlap in timing. For instance, such steps as heating and admixing are often separate, simultaneous, or partially overlapping in time in the art. Unless stated otherwise, when an element, material, or step capable of causing undesirable effects is present in amounts or in a form such that it does not cause the effect to an unacceptable degree it is considered substantially absent for the practice of this invention. Furthermore, the terms “unacceptable” and “unacceptably” are used to refer to deviation from that which can be commercially useful, otherwise useful in a given situation, or outside predetermined limits, which limits vary with specific situations and applications and may be set by predetermination, such as performance specifications. Those skilled in the art recognize that acceptable limits vary with equipment, conditions, applications, and other variables but can be determined without undue experimentation in each situation where they are applicable. In some instances, variation or deviation in one parameter may be acceptable to achieve another desirable end.

The term “comprising”, is synonymous with “including,” “containing,” or “characterized by,” is inclusive or open-ended and does not exclude additional, unrecited elements, material, or steps. The term “consisting essentially of” indicates that in addition to specified elements, materials, or steps; elements, unrecited materials or steps may be present in amounts that do not unacceptably materially affect at least one basic and novel characteristic of the subject matter. The term “consisting of” indicates that only stated elements, materials or steps are present.

The polyether NOPs of the present invention are made by reacting an initiator with a natural oil or derivative thereof, preferably a vegetable oil based (VOB) monomer such as is described in WO2004096882 which is hereby incorporated herein by reference to the fullest extent permitted by law and which also represents the knowledge of those skilled in the art with respect to the VOB. In the practice of the invention, the initiator has at least one active hydrogen, which is reacted with the VOB monomer, and has sufficient ether groups to render it more compatible or miscible with water, conventional polyether polyols or a combination thereof or to improve processibility or physical properties as previously described (collectively “desired results”). Such initiators are referred to herein as polyether initiators including amine tipped polyethers. It has been found that at least one of the desired results are advantageously accomplished using a NOP made with an initiator or combination of initiators having an average equivalent weight of at least about 480 per active hydrogen group, preferably at least about 600, more preferably at least about 700 most preferably at least about 1000 average equivalent weight per active hydrogen and optionally at most about 3000, preferably at most about 2500, more preferably at most about 2000 most preferably at most about 1500 average equivalent weight per active hydrogen. Thus, natural oil moieties are preferably separated by a molecular structure having an average molecular weight of advantageously at least about 1250 Daltons, preferably at least about 1300, more preferably at least about 1400, most preferably at least about 2000 and optionally at most about 6,000, preferably at most about 5000, more preferably at most about 4000 most preferably at most about 3000 Daltons. The initiator preferably has an active hydrogen functionality of preferably at least about 2 in one embodiment and preferably at least about 3 in another embodiment, and preferably at most about 8, more preferably at most about 6 and most preferably at most about 4.5.

In one embodiment, these characteristics are achieved using a single initiator, optionally with those impurities present in commercial products. In an alternative embodiment, the characteristics are achieved using combinations (referred to hereinafter as blends, mixtures or admixtures) of initiators in making the NOP or in combinations of NOP's. In either combination, advantageously at least about 10, more advantageously at least about 15, preferably at least about 20, more preferably at least about 25, most preferably at least about 30 weight percent (mass fraction) of the initiator used has an equivalent weight of at least about 480, with the same preferences previously listed for the initiator(s). When more than one initiator is used, the NOPs are optionally prepared separately with the resulting products combined in physical blends, used together in the same reaction to form insitu combinations, or a combination thereof.

The ether groups are preferably in poly(alkylene oxide) chains, more preferably in poly(propylene oxide) or poly(ethylene oxide) or a combination thereof, most preferably in a diblock structure of poly(propylene oxide) capped with poly(ethylene oxide).

The active hydrogen group is optionally any active hydrogen group that is sufficiently reactive to react with the natural oil or derivatives thereof under reaction conditions, preferably each active hydrogen group is independently a hydroxyl or amine group, more preferably a hydroxyl group, most preferably a primary hydroxyl group. In the case of amine groups, primary and secondary amines are preferred. Of the active hydrogen groups, advantageously at least about 50, more advantageously at least about 60, most advantageously at least about 70, preferably at least about 80, more preferably at least about 85, and most preferably at least about 90 and up to 100 mole percent of these groups are primary hydroxyl groups or amine groups, more preferably in each instance, primary hydroxyl groups. In a preferred embodiment, these preferred amounts of primary hydroxyl group in the initiator are reflected in the preferred amounts of primary hydroxyl group in the polyether NOP produced.

Thus preferred initiators are conveniently depicted by Formula 1:

R((OCH₂CHY)_{b}—XH)ₚ,

where Y is a H, CH₃ or higher alkyl group (preferably C1 to C16, more preferably C1 to C8, most preferably C1 to C4) or mixture thereof; X is an active hydrogen group, independently preferably O, N, or NH, most preferably O; p is 1 to 8, preferably 2 to 8; b is sufficient to result in an equivalent weight per active hydrogen group of at least about 480, preferably at least about 7 to a most about 70. The number of ether units in an arm of the polyether initiator, b, preferably at least about 9, most preferably at least about 12 when the equivalent weight is at least about 480, but at least about 13, more preferably at least about 14, most preferably at least about 15 when the equivalent weight is less than about 480; and regardless of equivalent weight, b is independently preferably at most about 70, more preferably at most about 55, most preferably at most about 45, such on average, the equivalent weight of the compound of Formula 1 is at least about 480, or on average each active hydrogen is separated from each other active hydrogen by an average of 19 ether groups (—OCH₂CHY—), preferably both. In the formula, each X is optionally the same or different. The initiator, therefore, encompasses polyols, polyamines and aminoalcohols. R generally represents a linear, cyclic chain or combination thereof of alkane (C—C), alkene (C═C), ether (C—O—C) linkages or combinations thereof. R preferably has at least about 1, more preferably at least about 2, most preferably at least about 3, and independently preferably has at most about 36, more preferably at most about 24, most preferably at most about 12 carbon atoms. The carbon atoms within the aforementioned chain are optionally substituted with a methyl or ethyl group. It should be noted that the value of each b in a polyether initiator optionally is the same or varies from one OCH₂CHY)_{b}—XH chain or “arm” of the polyether initiator to another. Furthermore, those skilled in the art will recognize that there will be variations in the numbers of alkylene oxide molecules added to a molecule in a reaction, thus in the value of b with in a molecule of polyether initiator and among molecules prepared simultaneously. To allow for variations, preferred values of b previously listed are understood to be the average b over all chains of the polyether initiator or combination thereof.

The R group is optionally exemplified by polyol initiators for polyethers that include neopentylglycol; 1,2-propylene glycol; trimethylolpropane; pentaerythritol; sorbitol; sucrose; glycerol; alkanediols such as 1,6-hexanediol; 2,5-hexanediol; 1,4-butanediol; 1,4-cyclohexane diol; ethylene glycol; diethylene glycol; triethylene glycol; 9(1)-hydroxymethyloctadecanol, 1,4-bishydroxymethylcyclohexane; 8,8-bis(hydroxymethyl)tricyclo[5,2,1,02,6]decene; Dimerol alcohol (36 carbon diol available from Henkel Corporation); hydrogenated bisphenol; 9,9(10,10)-bishydroxymethyloctadecanol; 1,2,6-hexanetriol; any of the aforementioned where at least one of the alcohol or amine groups present therein has been reacted with ethylene oxide, propylene oxide or mixture thereof; and combination thereof.

Exemplary polyamines that can form the R group of Formula 1 include ethylene diamine; neopentyldiamine, 1,6-diaminohexane; bisaminomethyltricyclodecane; bisaminocyclohexane; diethylene triamine; bis-3-aminopropyl methylamine; and triethylene tetramine. Exemplary aminoalcohols include ethanolamine, diethanolamine, and triethanolamine. Other compounds that are optionally used include polyols, polyamines or aminoalcohols described in U.S. Pat. Nos. 4,216,344; 4,243,818 and 4,348,543 and British Pat. No. 1,043,507.

Preferably, the initiator that forms R is selected from the group consisting of neopentylglycol; trimethylolpropane; pentaerythritol; sorbitol; sucrose; glycerol; 1,2-propylene glycol; 1,6-hexanediol; 2,5-hexanediol; 1,6-hexanediol; 1,4-cyclohexane diol; 1,4-butanediol; ethylene glycol; diethylene glycol; triethylene glycol; bis-3-aminopropyl methylamine; ethylene diamine; diethylene triamine; 9(1)-hydroxymethyloctadecanol; 1,4-bishydroxymethylcyclohexane; 8,8-bis(hydroxymethyl)tricyclo[5,2,1,02,6]decene; Dimerol alcohol; hydrogenated bisphenol; 9,9(10,10)-bishydroxymethyloctadecanol; 1,2,6-hexanetriol; any of the aforementioned where at least one of the alcohol or amine groups present therein has been reacted with ethylene oxide, propylene oxide or mixture thereof; and combination thereof.

More preferably the initiator that forms R is selected from the group consisting of neopentylglycol; 1,2-propylene glycol; trimethylolpropane; pentaerythritol; ethoxylated pentaerythritol; propoxylated pentaerythritol; sorbitol; sucrose; glycerol; ethoxylated glycerol; propoxylated glycerol; diethanolamine; alkanediols such as 1,6-hexanediol, 1,4-butanediol; 1,4-cyclohexane diol; 2,5-hexanediol; ethylene glycol; diethylene glycol, triethylene glycol; bis-3-aminopropyl methylamine; ethylene diamine; diethylene triamine; 9(1)-hydroxymethyloctadecanol, 1,4-bishydroxymethylcyclohexane; 8,8-bis(hydroxymethyl)tricyclo[5,2,1,02,6]decene; dimerol alcohol; hydrogenated bisphenol; 9,9(10,10)-bishydroxymethyloctadecanol; 1,2,6-hexanetriol and combination thereof.

Even more preferably the initiator that forms R is selected from the group consisting of glycerol; ethylene glycol; 1,2-propylene glycol; trimethylolpropane; bis-3-aminopropyl methylamine; ethylene diamine; pentaerythritol; diethylene triamine; sorbitol; sucrose; or any of the aforementioned where at least one of the alcohol or amine groups present therein has been reacted with ethylene oxide, propylene oxide or mixture thereof; and combination thereof.

Most preferably the initiator that forms R is glycerol, trimethylolpropane, bis-3-aminopropyl methylamine; pentaerythritol, glycerol or pentaerthritol that has been ethoxylated or propoxylated or a mixture thereof, sucrose, sorbitol or a mixture thereof.

Then, to form the polyether initiator, each active hydrogen group present therein is reacted with at least one alkylene oxide, preferably ethylene oxide or propylene oxide or a combination thereof; and most preferably a block of propylene oxide followed by a block of ethylene oxide, to form a polyether polyol by means within the skill in the art. The polyether polyol is preferably used as a polyol for reaction with the natural oil or derivative thereof. Alternatively the polyol is reacted by means within the skill in the art to convert one or more hydroxyl groups to alternative active hydrogen groups. In this case, the most preferred alkylene oxide is propylene oxide.

The polyether initiator is reacted with at least one natural oil or derivative thereof, preferably at least one vegetable oil based (VOB) monomer such as is described in WO2004096882. The natural oil or derivative thereof is optionally any natural oil or derivative of a natural oil reactive with at least one active hydrogen group on a polyether initiator according to the practice of the invention. Preferably the natural oil or derivative thereof has at least one acid, anhydride, acid chloride, or ester group reactive with at least one active hydrogen group on a polyether initiator to form at least one ester or amide. The natural oils or derivatives thereof are exemplified by VOB monomers herein, but the exemplification is not intended to limit the invention to the VOB monomers.

The VOB monomer or other fatty acid or derivative thereof is optionally formed from of any animal fat or vegetable oil that is comprised of triglycerides that upon saponification with a base such as aqueous sodium hydroxide yields a fatty acid and glycerol, where at least a portion of the fatty acids are preferably unsaturated fatty acids (that is, contain at least one carbon double bond). Preferred vegetable oils are those that yield at least about 70 percent unsaturated fatty acids by weight. More preferably, the vegetable oil yields at least about 85 percent, more preferably at least 87 percent, and most preferably at least about 90 percent by weight unsaturated fatty acids. It is understood that specific fatty acids derivable from a vegetable oil, animal fat or any other source are optionally used. That is to say, for example, palmitoleic, oleic, linoleic, linolenic and arachidonic fatty acid alkyl esters are optionally used to form the VOB monomer directly. It is preferred, however, to use a vegetable oil as previously described. Preferred vegetable oils include, for example, soy, safflower, cotton, linseed, peanut, olive, sunflower, canola, rapeseed, corn, palm oil or combination thereof. More preferably, the vegetable oil is a soy, sunflower, canola, corn, rapeseed oil, or combination thereof. Most preferably, the vegetable oil is soy, sunflower, canola oil or combination thereof. It is understood that the vegetable oil is optionally obtained from a genetically modified organism, such as genetically modified soybean, sunflower or canola.

Unsaturated fatty acid alkyl esters then are preferably formed, by any suitable process such as those known in the art, into preferred VOB monomers. For example, the hydroxymethyl group is optionally introduced by a hydroformylation process using a cobalt or rhodium catalyst followed by the hydrogenation of the formyl group to obtain the hydroxymethyl group by catalytic or by chemical reduction. Procedures to form the hydroxymethyl esters are described in U.S. Pat. Nos. 4,216,343; 4,216,344; 4,304,945 and 4,229,562 and in particular U.S. Pat. No. 4,083,816. Other known processes to form hydroxymethyl esters from fatty acids may also be used such as described by U.S. Pat. Nos. 2,3324,849 and 3,787,459.

In forming preferred VOB monomers, fatty acid alkyl esters are optionally completely formylated or only partially formylated. That is to say, the fatty acid alkyl esters of the particular vegetable oil optionally have some remaining unsaturated (C═C) bonds. Preferably, however, the amount of unsaturated bonds remaining after formylation is as described in WO2004096744, incorporated herein by reference to the extent permitted by law. After the fatty acid alkyl esters are formylated they are hydrogenated, such that there is desirably essentially no remaining unsaturated bonds (that is, trace amounts at most and preferably no detectable amounts of unsaturation).

At least one natural oil or derivative thereof and at least one polyether initiator are reacted together by any suitable means such as those known in the art to form at least one polyether NOP. For example, the method taught in WO200496882 and WO2004096883, which are incorporated herein by reference to the extent permitted by law and which represent the knowledge of those skilled in the art. The natural oil moiety is optionally reacted with the initiator before or after functionalization, that is, formation or introduction of hydroxyl groups or their precursors to the fatty acid moieties.

In a preferred embodiment, a functionalized natural oil moiety is formed, and then is reacted with a polyether initiator by any means within the skill in the art, for instance, transesterification, wherein an ester linkage is formed by reaction of a polyether initiator with the methyl ester of a functionalized fatty acid or, alternatively by esterification of an acid, chloride or anhydride form of the natural oil or derivative. The natural oil moiety of this embodiment is optionally functionalized by any means within the skill in the art, for example by epoxidation (and ring opening), amination, reacting with such compounds as maleic anhydride or perchloric acid, air oxidation, ozonolysis, hydroformylation, reaction with water such as blown oils where moist air in the presence of a catalyst preferably by epoxidation or hydroformylation.

In an alternative embodiment, the VOB is an unsaturated fatty acid unit in the acid form or in the methyl ester form. This monomer unit is optionally reacted with the polyether initiator (or combination thereof) using the same chemistry used for reaction with the functionalized VOB. After this VOB is reacted with the polyether initiator; it is then functionalized by any reaction within the skill in the art, such as those listed for functionalization before reaction with the polyether initiator. The functional group is directly useful for the formation of polyurethanes, or optionally undergoes further chemical reaction to form a useful functional group, such as the ring opening of an epoxy functional group to form the a NOP useful for such purposes.

The resulting polyol of the invention, referred to herein as a polyether NOP comprises at least two natural oil moieties separated by a molecular structure having at least about 19 ether groups or having an equivalent weight of at least about 480, preferably both. When the polyether initiator has more than 2 active hydrogen groups reactive with the natural oil or derivative thereof, each natural oil moiety is separated from another by an average of at least about 19 ether groups or a structure of molecular weight of at least about 480, preferably both. NOP's within the current skill in the art and not according to this invention are referred to herein as conventional NOP's.

Thus, preferred polyether NOP's are represented by Formula 2:

R(OCH₂CHY)_{b} XQ)ₚ,

wherein R, X, b, and p are as defined for Formula 1 and each Q independently represents at least one natural oil moiety. The Q's of a molecule are optionally the same or different. Q advantageously has the structure of at least one natural oil or, preferably, of one or more, fatty acids or derivatives thereof, more preferably at least one hydroxy functional fatty acid or derivative thereof, most preferably at least one hydroxymethyl methyl fatty acid or derivative thereof. More preferably Q represents a series of fatty acid derivatives, most preferably oligomerized by esterification or transesterification of at least one hydroxyl group or ester group, preferably the hydroxyl of a hydroxymethyl group on each fatty acid derivative with the acid or ester (preferably methyl ester) of another fatty acid derivative molecule or molecular portion. Preferably at least about an average of 0.5, more preferably at least about 0.8, most preferably at least about 1 fatty acid are oligomerized to form each natural oil moiety, Q. The number of fatty acid or fatty acid derivatives in each Q is preferably at most about 8, more preferably at most about 5, most preferably at most about 3.

The resulting polyether NOP is optionally used to make polyurethanes by reacting it with a polyisocyanate such as those known in the art using known methods to make such polyurethanes. Preferably the polyurethane is in the form of a flexible foam, rigid foam or elastomer. The polyether NOP is optionally used with or without conventional non NOP polyol or conventional NOP. When used with at least one conventional non NOP, the amount of polyether NOP or combined polyether NOP's is preferably at least about 1, more preferably at least about 20 most preferably at least about 50 and optionally at most about 100, preferably at most about 95, more preferably at most about 90 and most preferably at most about 80 weight percent of total polyols used with one or more non NOP making up the remainder. In an alternative embodiment, at least one polyether NOP is optionally used with at least one NOP which is not an NOP of this invention and they are optionally used with or without at least one conventional polyol. When at least one conventional NOP is used with at least one polyether NOP according to the practice of the invention in the absence of at least one conventional polyether polyol, the amount of polyether NOP or combined polyether NOP's is preferably at least about 5, more preferably at least about 10, most preferably at least about 20, and optionally at most about 95, preferably at most about 90, more preferably at most about 85 and most preferably at most about 80 weight percent of total polyols used, with at least one NOP which is a conventional NOP making up the remainder. In the alternative embodiment wherein at least one polyether NOP is used with at least one conventional polyol and at least one NOP that is a conventional NOP, the amount of polyether NOP or combined polyether NOP's is preferably at least about 5, more preferably at least about 10, most preferably at least about 20 and optionally at most about 95, preferably at most about 90, more preferably at most about 85 and most preferably at most about 80 weight percent of total polyols used, while the conventional polyol or combination thereof is preferably at least about 5, more preferably at least about 10, most preferably at least about 20 and optionally at most about 95, preferably at most about 90, more preferably at most about 85 and most preferably at most about 80 weight percent of total polyols used, with at least one polyether NOP according to the practice of the invention making up the remainder. Conventional NOP polyols include previously taught NOP's having initiators with polyether chains but having equivalent weights less than about 480.

Especially for the purpose of making foams, preferably molded foams, more preferably HR molded foams, a polyether NOP of the invention or combination thereof preferably has sufficient reactivity to react with the selected isocyante or combination thereof such that foams having desirable properties are produced. To this end, when the functional groups are all hydroxyl groups, advantageously an average of at least about 75, more advantageously an average of at least about 76, most advantageously an average of at least about 77, preferably an average of at least about 78, more preferably an average of at least about 79, most preferably an average of at least about an average of at least about 80 percent of the number of hydroxyl groups on the polyether NOP are primary hydroxyl groups.

Polyether NOP's of the invention are optionally used with any of the additives commonly known in the art for the production of polyurethane polymers. Any of a range of additives such as blowing agents, catalysts, surfactants, cell openers, colorants, fillers, load bearing enhancement additives such as copolymer polyols, water, internal mold releases, antistatic agents, antimicrobial agents, and other additives known to those skilled in the art are useful within the scope of the invention.

The NOP polyols of the invention are most useful in formulated systems that include an amount of natural oil polyol and a relatively high level of polyether polyols, especially ethylene oxide capped polyether polyols. For example, in HR molded foams, the compatibility of the formulated blends used to make the foam is superior and physical properties are improved. Physical property advantages are also seen in HR slabstock foams. Physical property advantages include higher resiliency, higher comfort factors, better tensile, tear or elongation. In each instance, the formulation comprising a polyether NOP of the invention is compared with the same formulation except that an operably comparable conventional NOP polyol is used in place of the NOP polyol of the invention and with both compared formulations prepared and processed the same way. An operably comparable conventional NOP is an NOP within the current skill in the art and not according to the present invention, which conventional NOP has properties that would indicate that one skilled in the art would reasonably expect it to function similarly in making the type of article for which the formulation is intended (for instance, an HR molded foam). These properties include an equivalent weight within about 15, preferably within about 10 weight percent of the polyether NOP; and independently preferably include at least one of (a) an average functionality within about 1 unit, more preferably within about 0.7 units, of the polyether NOP; or (b) an average percentage of primary active hydrogen groups preferably within about 2 percentage points, more preferably within about 1 percentage points of that of the polyether NOP; preferably both (a) and (b).

It also has been found that the polyether NOP's of this invention are useful in polyurethane foams made with a wide range of water concentrations. Generally, the water concentrations may range from about 0 part per hundred parts to about 10 parts per hundred parts of polyol by weight. Preferably, the water concentration is at least about 2, more preferably 3 and most preferably at least about 4 to preferably at most about 9, more preferably at most 8 and most preferably at most about 6 parts per hundred parts of polyol by weight.

Notably, NOP's or mixtures of NOP's wherein the natural oil moieties are separated by a molecular structure having at least about 19 ether groups or an equivalent weight of at least about 480, as compared with an operably comparable (as previously described) conventional NOP having natural oil moieties separated by a molecular structure of lower equivalent weight or one not having ether groups, achieve at least one of (a) greater miscibility or compatibility with conventional polyether polyols, (b) greater miscibility or compatibility with water, (c) greater processability in making a polyurethane, preferably a polyurethane foam, (d) increased tensile strength in a polyurethane; (e) increased elongation in a polyurethane, or (f) increased resiliency in a polyurethane, where, in each instance, the comparative polyurethanes are produced by the same method using the same formulation except that the polyether NOP of the invention are replaced by an operably comparable conventional NOP, preferably including at least one of (a) or (c) or, more preferably both (a) and (c). Processability improvements are indicated by at least one of: a broader tin latitude (defined as the range of tin catalyst concentration over which good quality foams can be made), better foam flowability, higher open cell content and, in a molded application, a lower force to crush after the foam is made.

Objects and advantages of this invention are further illustrated by the following examples. The particular materials and amounts thereof, as well as other conditions and details, recited in these examples should not be used to limit this invention. Rather they are illustrative of the whole invention. Unless stated otherwise all percentages, parts and ratios are by weight. Examples of the invention are numbered while comparative samples, which are not examples of the invention, are designated alphabetically.

### Examples

The following materials are used in the examples of the invention:
NO-1 is a hydroformylated, hydrogenated fatty acid methyl ester mixture derived from soy bean oil as taught in Example 1 for hydroformylation and Example 17 for hydrogenation of WO2004096744 which is incorporated by reference herein
INT-1 is a glycerin initiated poly(ethylene oxide) having a hydroxyl number of 267 and a molecular weight of approximately 620.
INT-2 is a mutifunctional polyether polyol having a nominal functionality of 4.2, a hydroxyl number of 31, and an average equivalent weight of 1810 commercially available from The Dow Chemical Company under the trade designation Specflex™ NC630.
INT-3 is a trifunctional polyether polyol having hydroxyl number of 48, and an average equivalent weight of 1168 commercially available from The Dow Chemical Company under the trade designation Voranol™ 3512.
INT-4 is a trifunctional polyether polyol having a hydroxyl number of 36 and an average equivalent weight of 1558 commercially available from The Dow Chemical Company under the trade designation DWS4002.
INT-5 is a polyether polyol initiated from glycerin, propoxylated with propylene oxide using a KOH catalyst to a molecular weight of 3700 g/mol, and subsequently reacted with EO using a KOH catalyst to reach a molecular weight of 4600 g/mol.
PEPO-1 is a copolymer polyol, having solid content of 40 weight percent and hydroxyl number of 22 commercially available from The Dow Chemical Company under the trade designation Specflex™ NC 701.
PEPO-2 is a sucrose/glycerin co-initiated polyol, having 75 percent ethylene oxide by weight and an equivalent weight of 1795 commercially available from The Dow Chemical Company under the trade designation Voranol™ 4053.
PEPO-3 is a copolymer polyol, having solid content of 40 weight percent and hydroxyl number of 30 commercially available from The Dow Chemical Company under the trade designation VORANOL™ 3943A
CAT-1 is a crosslinker, diethanol amine (DEOA), commercially available from The Dow Chemical Company.
CAT-2 is an amine catalyst commercially available from Air Products and Chemicals under the trade designation Dabco™ BLV.
CAT-3 is a silicone surfactant commercially available from GE Silicones under the trade designation Niax™ U2000.
CAT-4 is Tin catalyst commercially available from Air Products and Chemicals under the trade designation T-12.
CAT-5 is a silicone surfactant commercially available from Goldschmidt Chemical under the trade designation Tegostab™ B-8715.
CAT-6 is a silicone surfactant commercially available from GE Silicones under the trade designation Niax™ L2125.
CAT-7 is glycerin.
NCO-1 is toluene diisocyanate which is a 80:20 mixture by weight of the 2,4 and 2,6 isomers of toluene diisocyanate, respectively, commercially available from The Dow Chemical Company under the trade designation T-80. Water is deionized water.

### Examples 1 to 5 and Comparative Sample A

### Preparation of Polyols

### Comparative Sample A: Preparation of Polyol-1

NO-1 (34092 g) is charged to a 30 gallon (114 l) stainless steel jacketed reactor vessel together with INT-1 initiator (15595 g). The reactor vessel is equipped with a nitrogen sparger, a turbine for gas dispersion, a vacuum system, and hot oil as a heating medium. The mixture is devolatilized by heating to 150° C. under 500 mmHg (66.7 kPa) and a nitrogen flow (1.0 standard cubic feet per minute, scfm (0.47 standard l/s)). The speed of the agitator is set at 200 rpm. Tin ethylhexanoate (24.84 g) is added, and the reaction mixture is heated to 195° C. under atmospheric pressure and a nitrogen flow of 1.2 scfm (0.57 standard l/s). The pressure is reduced to 500 mmHg (66.7 kPa) and the reaction is continued for another 2 hours. Polyol-1 having a hydroxyl number of 91 and a viscosity of 2000 centipoise (cP) at 25° C. is obtained. The procedure of ASTM 4274 is used to determine hydroxyl number. The procedure of ASTM D4878 is used to determine viscosity.

### Example 1

### Preparation of Polyol-3

The procedure of Comparative Sample A is repeated except using NO-1 (11885.5 g), tin 2-ethylhexanoate (hereinafter referred to as tin catalyst) (27.50 g), and INT-2 (37686 g) and INT-1 (5428.5 g) as co-initiators. Polyol-3 having a hydroxyl number of 46 and a viscosity of 1650 CP at 25° C. is obtained.

### Example 2

### Preparation of Polyol-4

The procedure of Comparative Sample A is repeated except using NO-1 (14342.6 g), tin catalyst (27.5 g), and INT-2 (34107.6 g) and INT-1 (6549.9 g) as co-initiators. Polyol-4 having a hydroxyl number of 51 and a viscosity of 1710 cP at 25° C. is obtained.

### Example 3

### Preparation of Polyol-5

The procedure of Comparative Sample A is repeated except using NO-1 (18078.5 g), and tin catalyst (27.5 g), and INT-2 (28666 g) and INT-1 (8255.5 g) as co-initiators. Polyol-5 having a hydroxyl number of 57 and a viscosity of 1770 cP at 25° C. is obtained.

### Example 4

### Preparation of Polyol-8

The procedure of Comparative Sample A is repeated except using NO-1 (9619.5 g), tin catalyst (27.5 g), and INT-2 as initiator (45380.5 g). Polyol-8 having a hydroxyl number of 25 and a viscosity of 2080 cP at 25° C. is obtained.

### Example 5

### Preparation of Polyol-10

The procedure of Comparative Sample A is repeated except using NO-1 (29848 g), tin catalyst (27.8 g), and INT-4 (12170 g) and INT-1 (13628 g) as co-initiators. Polyol-10 having a hydroxyl number of 77 and a viscosity of 1680 cP at 25° C. is obtained.

### Example 6

### Preparation of Polyol-12

The procedure of Comparative Sample A is repeated except using NO-1 (13000 g), tin catalyst (26.58 g), and INT-5 as initiator (40150 g). Polyol-12 having a hydroxyl number of 29 and a viscosity of 1770 cP at 25° C. is obtained.

### Example 7

### Preparation of Polyol-13

The procedure of Comparative Sample A is repeated except using NO-1 (16000 g), tin catalyst (26.74 g), and INT-5 as initiator (37470 g). Polyol-13 having a hydroxyl number of 27 and a viscosity of 2130 cP at 25° C. is obtained.

### Example 8

### Preparation of Polyol-14

The procedure of Comparative Sample A is repeated except using NO-1 (14013 g), tin catalyst (26.98 g), and INT-2 as initiator (39952 g). Polyol-14 having a hydroxyl number of 24 and a viscosity of 2780 cP at 25° C. is obtained.

Thus, the following polyols are obtained for examples 1-13:
Polyol-1: Natural oil polyol prepared from transesterification of NO-1 and INT-1, having a hydroxyl number of 91 and a viscosity of 2000 centipoise (cP) at 25 degrees Celsius. See Comparative Sample A. Polyol-1 is not an example of the invention because the initiator provides a separation between natural oil moieties of only about 207 equivalent weight per natural oil moiety.
Polyol-2: Physical blend of 30 weight percent polyol 1 and 70 weight percent INT-2, having a calculated hydroxyl number of 46.
Polyol-3: Natural oil polyol in situ blend prepared from transesterification of NO-1, INT-1 and INT-2, having a hydroxyl number of 46 and a viscosity of 1650 cP. See Example 1. Polyol-3 is an example of the invention because INT-2 provides an average separation between natural oil moieties of about 1810 equivalent weight per natural oil moiety.
Polyol -4: Natural oil polyol prepared from transesterification of NO-1, INT-1 and INT-2, having a hydroxyl number of 51 and a viscosity of 1710 cP. See Example 2. Polyol-4 is an example of the invention because INT-2 provides an average separation between natural oil moieties of about 1810 equivalent weight per natural oil moiety.
Polyol-5: Natural oil polyol prepared from transesterification of NO-1, INT-1 and INT-2, having a hydroxyl number of 57 and a viscosity of 1770 cP. See Example 3. Polyol-5 is an example of the invention because the INT-2 provides an average separation between natural oil moieties of about 1810 equivalent weight per natural oil moiety.
Polyol-6: Physical blend of 82.5 weight percent Polyol 4 and 17.5 weight percent of INT-2, having a calculated hydroxyl number of 46.
Polyol-7: Physical blend of 65 weight percent of Polyol 5 and 35 weight percent of INT-2, having a calculated hydroxyl number of 46.
Polyol-8: Natural oil polyol prepared from transesterification of NO-1 and INT-2, having a hydroxyl number of 25 and a viscosity of 2080 cP. See Example 4. Polyol-8 is an example of the invention because INT-2 provides an average separation between natural oil moieties of about 1810 equivalent weight per natural oil moiety.
Polyol-9: Physical blend of 40 weight percent Polyol-1 and 60 weight percent INT-3, having a calculated hydroxyl number of 50.6.
Polyol-10: Natural oil polyol prepared from the transesterification of NO-1, INT-1 and INT-4, having a hydroxyl number of 77 and a viscosity of 1680 cP. See Example 5. Polyol-10 is an example of the invention because the INT-4 provides an average separation between natural oil moieties of about 1558 equivalent weight per natural oil moiety.
Polyol-11: Physical blend of 52 weight percent Polyol-10 and 48 weight percent INT-3, having a calculated hydroxyl number of 63.
Polyol-12: Natural oil polyol prepared from transesterification of NO-1 and INT-5, having a hydroxyl number of 29 and a viscosity of 1770 cP. See Example 6. Polyol-12 is an example of the invention because INT-2 provides an average separation between natural oil moieties of about 1530 equivalent weight per natural oil moiety.
Polyol-13: Natural oil polyol prepared from transesterification of NO-1 and INT-5, having a hydroxyl number of 27 and a viscosity of 2130 cP. See Example 7. Polyol-13 is an example of the invention because INT-2 provides an average separation between natural oil moieties of about 1530 equivalent weight per natural oil moiety.
Polyol-14: Natural oil polyol prepared from transesterification of NO-1 and INT-2, having a hydroxyl number of 24 and a viscosity of 2780 cP. See Example 8. Polyol-14 is an example of the invention because INT-2 provides an average separation between natural oil moieties of about 1810 equivalent weight per natural oil moiety.

### Example 9

### Compatibility of Natural Oil Polyols

Each blended polyol (Polyol-2, Polyol-3, Polyol-6, Polyol-7, Polyol 9, and Polyol 11) is stored for a period of two weeks after being prepared, whether prepared by physical blending or by in situ preparation at a temperature of 23° C. and atmospheric pressure in a closed glass bottle. After the 2 week period, each blend is visually observed for haziness and phase separation, indicating incompatibility of components of the blend. Lack of haze is referred to as clear; lack of phase separation as stable. The components of clear and stable blends are interpreted to be compatible. The components of Polyol-2 are observed to be not compatible (hazy and phase separated in 2 weeks). Similarly, the components of Polyol-9 are observed to be not compatible. This is consistent with the fact that Polyol-1, the NOP in both these blends is not an example of the invention. The components of the blends referred to as Polyol-3, Polyol-6, Polyol-7, and Polyol-11 are observed to be compatible, that is they are clear and stable after 2 weeks. These observations are consistent with the fact that the NOP in each instance is an example of the invention.

### Comparative Sample B and Examples 10-12

### Foams from Polyols 1, 12, 13 and 14, Respectively

Foams are formed from Polyols 1, 12, 13 and 14 using a high pressure impingement mixing foam machine commercially available from Cannon USA under the trade designation Cannon A 40 having a TDI/ISO tank, a polyol tank, and a mixing head commercially available from the same source under the trade designation Cannon FLP-HP-14 mixing head. Toluene diisocyanate (TDI) is pumped directly into the TDI/ISO tank and is conditioned to 78° F. (26° C.). The polyol side components (that is, everything except the isocyanate reactant or combination thereof) indicated in Table 1 are weighed into a blending vessel and mixed. The resulting polyol blend is then pumped to the polyol tank and is conditioned to 78° F. (26° C.). Once the component temperatures have stabilized at 78° F. (26° C.) foam pads are produced using the mixing head. The mixing head pressure settings are 137 Bar for the 0.8 mm ISO orifice and 170 Bar for the 1.0 mm polyol orifice. The components are metered to meet the desired parts by weight indicated in Table 1. The components are mixed through using high pressure impingement mixing and poured into the test mold using the mixing head at the previously outlined conditions. Physical property foam pads (15″×15″×4.5″) (38×38×11 cm) are produced at a mold temperature of 150° F. (66° C.) and with a curing time of 6 minutes. When demolded, the parts are weighed and then immediately crushed to open the cell windows. The foams are crushed 3 times. First at 2 inches (5.08 cm), next at 1½ inches (3.81 cm) and finally at 1 inch (2.54 cm) to insure that the foams do not split. Foam samples are then allowed to condition for seven days before being cut for physical property testing according to the procedures of ASTM D 3574 to give the results shown in Table 2.

TABLE 1
FORMULATION FOR FOAMS OF COMPARATIVE
SAMPLE (CS) B and EXAMPLES (EX) 10-13

| | Formulation | Formulation | Formulation | Formulation | Formulation |
|---|---|---|---|---|---|
| | 1-CS B* | 2-EX 10 | 3-EX 11 | 4-EX 12 | 5-EX 13 |
| PEPO-2 | 50 | 0 | 0 | 0 | 0 |
| Polyol-1 | 30 | 0 | 0 | 0 | 0 |
| PEPO-3 | 20 | | | | |
| Polyol 12 | | 80 | 70 | | |
| Polyol 13 | | | | 70 | |
| Polyol 14 | | | | | 70 |
| PEPO-1 | | 20 | 30 | 30 | 30 |
| CAT-1 | 0.7 | 1.6 | 1.6 | 0.8 | 0.8 |
| CAT-2 | 0.3 | 0.4 | 0.4 | 0.4 | 0.04 |
| CAT-3 | 0.05 | 0.08 | 0.08 | 0.04 | 0.04 |
| CAT-4 | 0.1 | 0.1 | 0.1 | 0.1 | 0.1 |
| CAT-5 | 0.8 | | | | |
| CAT-6 | | 1 | 1 | 1 | 1 |
| CAT-7 | | | | 1 | 1 |
| Water | 3.5 | 3.5 | 3.5 | 3.5 | 3.5 |
| NCO-1 | | | | | |
| Index | 85 | 100 | 100 | 100 | 100 |
| Formulation | Not stable, | Stable for | Stable for | Stable for | Stable for |
| stability | phase | more than 2 | more than 2 | more than 2 | more than 2 |
| | separate | weeks | weeks | weeks | weeks |
| | into two | | | | |
| | layers in 4 | | | | |
| | hours | | | | |

*Not an example of the invention.

TABLE 2
PROPERTIES OF THE FOAMS OF EXAMPLES 10-13

| | | Foam | Foam | Foam | Foam | Foam |
|---|---|---|---|---|---|---|
| Physical property (unit) | Method of | 1 | 2 | 3 | 4 | 5 |
| | determination | CS B | EX 10 | EX 11 | EX 12 | EX 13 |
| Air flow (cu ft/min) | ASTM 3574 G | 0.8 | 0.4 | 0.4 | 1.2 | 1.5 |
| Air flow (liters/sec) | conversion | 1.4 | 0.7 | 0.7 | 2.0 | 2.5 |
| DENSITY (lb/cubic ft) | ASTM 3574-01 test A | 1.95 | 2 | 2 | 2.05 | 2.1 |
| DENSITY kg/m³ | conversion | 31.2 | 32.0 | 32.0 | 32.8 | 33.6 |
| 75% dry compression | ASTM 3574-01 test | 21 | 18 | 17 | 10 | 11 |
| set (%) | | | | | | |
| 75% humid compression | ASTM 3574-01 test | 22 | 18 | 18 | 13 | 14 |
| set (%) | | | | | | |
| 25% IFD (lb) | ASTM 3574 | 24 | 26 | 31 | 28 | 30 |
| 25% IFD (N) | conversion | 107 | 116 | 138 | 125 | 133 |
| 65% IFD (lb) | ASTM 3574 | 64 | 68 | 78 | 79 | 87 |
| 65% IFD (N) | conversion | 285 | 302 | 347 | 352 | 387 |
| RESILIENCY (%) | ASTM 3574 H | 50.8 | 55.2 | 54.6 | 52 | 54.4 |
| Tensile strength (psi) | ASTM 3574 | 15 | 17 | 19 | 17 | 15 |
| Tensile strength (kPa) | conversion | 103 | 117 | 131 | 117 | 103 |
| Tear strength (lb/ft) | ASTM 3574 | 1.2 | 1.06 | 1.17 | 1.13 | 1.06 |
| Tear strength (N/m) | conversion | 17.7 | 15.6 | 17.2 | 16.6 | 15.6 |
| Elongation % | ASTM 3574 | 104 | 94 | 109 | 81 | 78 |

*Not an example of the invention.

These results show that NOP's wherein the natural oil moieties are separated by a molecular structure having ether groups and an equivalent weight of at least about 480, as compared with a NOP having natural oil moieties separated by a molecular structure of lower equivalent weight or one not having ether groups, achieve at least one of (a) greater miscibility or compatibility with conventional polyether polyols, (b) greater miscibility or compatibility with water, (c) increased tensile strength in a polyurethane; (d) increased elongation in a polyurethane, or (e) increased resiliency in a polyurethane as compared with a foam made using a NOP having a smaller polyether groups separating the natural oil moieties. The tear strengths in these Examples and Comparative Sample are considered comparable within experimental error.

### Comparative Samples C and D and Example 14

For Comparative Samples C and D (CS C and CS D) and Example 14 (EX 14) the following materials were used:
Polyol 12 Natural oil polyol prepared from transesterification of NO-1 and INT-5, having a hydroxyl number of 29 and a viscosity of 1770 cP. See Example 6. Polyol-12 is an example of the invention because INT-2 provides an average separation between natural oil moieties of about 1530 equivalent weight per natural oil moiety.
VORANOL*4820 A 4800 MW polyether polyol with 17% EO cap. Available from the Dow Chemical Company.
SPECFLEX*NC700 A grafted polyether polyol containing 40% copolymerized styrene and acrylonitrile (SAN). Available from The Dow Chemical Company.
BIOH Polyol 500 Renewable resource polyol. Available from Cargill.
VORANATE T80 A toluene diisocyanate (80% 2,4-toluene diisocyanate and 20% 2,6-toluene diisocyanate by weight) composition available from The Dow Chemical Company
ORTEGOL 204 A crosslinker blend of urea, sorbitol and 25% water. Available from Evonik Industrieso
NIAX L2100 A silicone surfactant available from Momentive Performance Materials.
KOSMOS 54 Zinc Ricinoleate based stabilization catalyst. Available from Evonik Industries.
NIAX A1 70% bis(2dimethyl aminoethyl)ether and 30% dipropylene glycol. Available from Momentive Performance Materials.
DABCO 33LV A 33% solution of triethylenediamine in propylene glycol available from Air Products & Chemicals Inc.
DEOA 85% Diethanolamine with 15% water.
KOSMOS 29 A stannous octoate catalyst. Available from Evonik Industries.

The test methods used are set forth in Table 3:

TABLE 3
Test methods

| Property | Units | Standard |
|---|---|---|
| Density | kg m⁻³ | ISO 845 |
| CFD 40% | kPa | ISO 3386 |
| IFD 40% | N | BS EN ISO 2439 |
| Tensile | kPa | ISO 1798 |
| Elongation | % | ISO 1798 |
| Resilience | % | ASTM D3574 |
| Airflow crushed | scfm | ASTM D3574 |
| Compression Set | % | ISO 1856 |
| 75% | | |
| Compression Set | % | ISO 1856 |
| 90% | | |
| Dynamic Fatigue | N | BS EN ISO 2439 |

Foams of approximately 1.0 kg are prepared by first mixing all the foam components except the tin catalyst and isocyanate for 20 seconds at 2500 rpm using a mixer. The tin catalyst is then added followed by additional mixing for 10 seconds at 2500 rpm. Finally, the isocyanate is added and mixed for 10 seconds at 2500 rpm. The mixture is then poured into a 25×25×20 box and allowed to rise. The full rise and blow off times are recorded. The foams are then transferred in to a 140° C. oven for 10 minutes. The formulations and test results of the foams are given in Table 4:

TABLE 4
Formulations used to produce foams

| Foam Ref | CS C | EX 14 | CS D |
|---|---|---|---|
| VORANOL4820 | 70 | | 55 |
| Polyol 12 | | 70 | |
| BiOH500 | | | 15 |
| SPECFLEXNC700 | 30 | 30 | 30 |
| ORTEGOL 204 | 1.80 | 1.80 | 1.80 |
| DEOA 85% | 0.60 | 0.60 | 0.60 |
| Water | 1.50 | 1.50 | 1.50 |
| NIAX A1 | 0.10 | 0.10 | 0.10 |
| DABCO 33LV | 0.15 | 0.15 | 0.15 |
| NIAX L2100 | 0.80 | 0.80 | 0.80 |
| KOSMOS 29 | 0.10 | 0.10 | 0.10 |
| KOSMOS 54 | 0.50 | 0.50 | 0.50 |
| TDI (T-80) | 30.6 | 29.8 | 31.3 |
| TDI Index | 110 | 110 | 110 |
| Density/kg m⁻³ | 47 | 49 | 46 |
| CFD 40/kPa | 3.3 | 3.4 | 3.5 |
| Air flow crushed/ | 4.5 | 3.9 | 4.2 |
| scfm | | | |
| Resilience/% | 56 | 56 | 44 |

To obtain comparative data, the index, water, catalyst, silicone and crosslinker levels are all kept constant for the three types of foams. The renewable content in the NOP foam is also kept constant (14%) and is calculated as the renewable carbon in the foam (carbon from plant sources) as a fraction of the total carbon in the foam11. It has been assumed the BiOH 500 polyol has 76.8% carbon of which 97% is renewable carbon.

The resilience of the foams based on VORANOL 4820 (CS C) and Polyol 12 (EX 14) is 56%, higher than 55% which may be used to define HR foams, in contrast the values obtained for the foam based on the BiOH 500 (CS D) blend is significantly lower at 44%, despite comparable foam hardness and airflow. The lower resilience measured for the foam based on the competitive NOP could be potentially due to increased phase mixing because of the lower equivalent weight of the polyol.

### Comparative Sample E and Example 15

For Comparative Sample E (CS E) and Example 15 (EX 15) the same materials are used as for Comparative Sample C and Example 14. However, foams are made using a continuous foaming process using a Polymech machine. The foam components are mixed with a high shear mixer at 4900 rpm and dispensed on a conveyor with a line speed of 3.7 m/min. The foam is cut into blocks and the top, center and bottom of each block is characterized. All foams are allowed to cure for seven days prior to physical property testing. The formulations and test results of the foams are given in Table 5:

TABLE 5
Foam property measurements

| Foam Ref | CS E | EX15 |
|---|---|---|
| VORANOL4820 | 70 | |
| Polyol 12 | | 70 |
| SPECFLEXNC700 | 30 | 30 |
| ORTEGOL 204 | 1.80 | 1.80 |
| DEOA 85% | 0.60 | 0.60 |
| Water | 1.50 | 1.50 |
| NIAX A1 | 0.10 | 0.10 |
| DABCO 33LV | 0.15 | 0.15 |
| NIAX L2100 | 0.80 | 0.80 |
| KOSMOS 29 | 0.10 | 0.10 |
| KOSMOS 54 | 0.50 | 0.50 |
| TDI (T-80) | 30.6 | 29.8 |
| TDI Index | 110 | 110 |
| Density/kg m⁻³ | 48.3 | 49.5 |
| CFD 40%/kPa | 3.2 | 3.0 |
| IFD 40%/N | 140 | 130 |
| SAG Factor | 2.8 | 3.1 |
| Tensile Strength/ | 92 | 84 |
| kPa | | |
| Elongation/% | 126 | 131 |
| Resilience/% | 61 | 60 |
| Air flow/scfm | 3.1 | 3.9 |
| Compression set | 3.7 | 4.4 |
| 75%/% | | |

Foams produced with Polyol 12 (EX 15) has comparable processing lattitude and cell structure to VORANOL 4820 foams (CS E) and the foam produced has no noteworthy color or odor. The tensile strength and elongation are comparable to the control foam. The recovery properties (resilience) measured are 61% and 60% for CS E and EX 15 respectively. The increase in resilience measured of foam made on the machine (60%) compared to the lab samples (56%) may be due to macro-mechanical (cellular architecture) differences and improved mixing.

### Embodiments of the Invention Include

1. A polyether NOP comprising at least two natural oil moieties separated by a molecular structure having an average of at least about 19 ether groups between any 2 of the natural oil moieties or a polyether molecular structure having an equivalent weight of at least about 480, said polyol referred to hereinafter as a polyether NOP.

2. A method of making the polyether NOP of Embodiment 1 comprising admixing at least one polyether initiator having an equivalent weight of at least about 480 or at least about 19 ether groups per active hydrogen group with at least one natural oil or derivative thereof under reaction conditions such that at least one polyether NOP is formed.

3. A polymer comprising the reaction product of Embodiment 1 and at least one other monomer different from a polyether NOP and reactive therewith to form a polymer, preferably at least one polyisocyanate.

4. A method of making a polymer comprising admixing at least one polyether NOP of Embodiment 1 with at least one monomer reactive therewith under reaction conditions such that a polymer is formed.

5. An article comprising at least one polymer of Embodiment 3.

6. A composition comprising at least one polyether NOP of Embodiment 1.

7. The composition of Embodiment 6 also comprising at least one polyisocyanate compound.

8. A composition comprising at least one polymer of Embodiment 3.

9. At least one foam, preferably at least one molded foam, more preferably at least one high resiliency foam, most preferably at least one molded high resiliency foam made from at least one polyether NOP of any of the other embodiments.

10. The foam of any other embodiment having at least one of higher resiliency, higher comfort factors, better tensile, tear or elongation compared a foam of the same formulation except that an operably comparable conventional NOP polyol is used in place of the polyether NOP of the invention and with both compared formulations prepared and processed the same way (that is, using the same process) wherein the operably comparable conventional NOP has an equivalent weight within about 15, preferably within about 10 weight percent of the polyether NOP; and preferably has at least one of (a) an average functionality within about 1 unit, more preferably within about 0.7 units, of that of the polyether NOP; or (b) an average percentage of primary active hydrogen groups preferably within about 2 percentage points, more preferably within about 1 percentage points of that of the polyether NOP; most preferably both (a) and (b).

11. A foam formed from at least one polyether NOP of any other embodiments wherein the foam forming formulation comprises from about 0, 2, 3, 4 or 10 to preferably at most about 9, 8, or 6 parts per hundred parts per hundred parts of water by weight based on total weight of polyols and water.

12. The polyether NOP, method, composition, polymer, polyurethane, article, or foam of any other embodiment wherein the polyether NOP is produced using a natural oil or derivative thereof to react with a polyether initiator.

13. The polyether NOP, method, composition, polymer, polyurethane, article, or foam of any other embodiment wherein the polyether initiator has an average equivalent weight of at least about any of 480, 600, 700 or 1000 per active hydrogen group and optionally at most about any of 3000, 2500, 2000, or 1500 average equivalent weight per active hydrogen.

14. The polyether NOP, method, composition, polymer, polyurethane, article, or foam of any other embodiment wherein polyether initiator has a molecular structure having an average molecular weight of advantageously at least about any of 960, 1200, or 1400 Daltons, and optionally at most about any of 6,000, 5000, 4000, or 3000 Daltons.

15. The polyether NOP, method, composition, polymer, polyurethane, article, or foam of any other embodiment wherein the polyether initiator has an active hydrogen functionality of at least about 2 or 3 and optionally at most about any of 8, 6 or 4.5.

16. The polyether NOP, method, composition, polymer, polyurethane, article, or foam of any other embodiment wherein a combination of polyether initiators is used in making the polyether NOP or in making a combination of NOPs that includes at least one polyether NOP, wherein at least one of the polyether initiators is present in an amount of at least about any of 10, 15, 20, 25 or 30, and optionally at most about 100, 95, 90, 85, or 80 weight percent.

17. The polyether NOP, method, composition, polymer, polyurethane, article, or foam of any other embodiment wherein the polyether NOP is present in an amount of at least about any of 10, 15, 20, 25 or 30, and optionally at most about 100, weight percent.

18. The polyether NOP, method, composition, polymer, polyurethane, article, or foam of any other embodiment wherein the polyether initiator comprises at least 2 poly(alkylene oxide) chains, more preferably in poly(propylene oxide) or poly(ethylene oxide) or a combination thereof, most preferably in a diblock structure of poly(propylene oxide) capped with poly(ethylene oxide), wherein preferably the alkylene oxide comprises at least about any of 5, 10, or 15 weight percent ethylene oxide.

19. The polyether NOP, method, composition, polymer, polyurethane, article, or foam of any other embodiment wherein the polyether initiator has an equivalent weight per ether oxygen of at most about any of 500, 250, or 100.

20. The polyether NOP, method, composition, polymer, polyurethane, article, or foam of any other embodiment wherein the polyether initiator has at least 2 active hydrogen groups each independently selected from a hydroxyl or amine group, more preferably a hydroxyl group, most preferably a primary hydroxyl group.

21. The polyether NOP, method, composition, polymer, polyurethane, article, or foam of any other embodiment wherein the polyether initiators is depicted by Formula 1: R((OCH₂CHY)_{b}—XH)ₚ, where Y is a H CH₃ or higher alkyl group (preferably C1 to C16, more preferably C1 to C8, most preferably C1 to C4) or mixture thereof; X is an active hydrogen group, preferably O, N, or NH, most preferably O; p is 1 to 8, preferably 2 to 8; b is sufficient to result in an equivalent weight per active hydrogen group of at least about 480, preferably at least about 7 to a most about 70. The number of ether units in an arm of the polyether initiator, b, is preferably at least about 7, more preferably at least about 9, most preferably at least about 12 when the equivalent weight is at least about 480, but at least about 13, more preferably at least about 19, most preferably at least about 15 when the equivalent weight is less than about 480; and regardless of equivalent weight, b is independently preferably at most about 70, more preferably at most about 55, most preferably at most about 45. In the formula, each X is optionally the same or different. R generally represents a linear, cyclic chain or combination thereof of alkane (C—C), alkene (C═C), ether (C—O—C) linkages or combinations thereof. The carbons within the aforementioned chain may be substituted with a methyl or ethyl group.

22. The polyether NOP, method, composition, polymer, polyurethane, article, or foam of any other embodiment wherein the natural oil or derivative thereof comprises at least one vegetable oil based (VOB) monomer, alkyl esters, more preferably methyl esters of the fatty acids, which have at least one hydroxyl group, more preferably resulting from reaction at sites of unsaturation, most preferably resulting from hydroformylation at sites of unsaturation; most preferably methyl esters of at least one fatty acid having at least one hydroxyl group, advantageously at least one primary hydroxyl group, preferably at least one hydroxymethyl group, more preferably having at least one hydroxymethyl group where the original fatty acid had a double carbon to carbon bond, most preferably having at least one hydroxymethyl group at each site of a double bond on the original fatty acid.

23. The polyether NOP, method, composition, polymer, polyurethane, article, or foam of any other embodiment wherein the VOB monomer is formed from of any animal fat or vegetable oil that is comprised of triglycerides, preferably where at least a portion of the fatty acids are unsaturated fatty acids, more preferably wherein at least about any of 70, 85, 87 or 90 percent unsaturated fatty acids by weight, more preferably selected from palmitoleic, oleic, linoleic, linolenic, arachidonic fatty acid alkyl esters and a combination thereof.

24. The polyether NOP, method, composition, polymer, polyurethane, article, or foam of any other embodiment wherein the polyether NOP is represented by Formula 2: R(OCH₂CHY)_{b} XQ)ₚ,

25. wherein R, X, b, and p are as defined for Formula 1 and each Q independently represents at least one natural oil moiety; more preferably Q represents a series of fatty acid derivatives, most preferably oligomerized by transesterification of at least one hydroxyl group, preferably the hydroxyl of a hydroxymethyl group on each fatty acid derivative with the acid or ester (preferably methyl ester) of another fatty acid derivative molecule or molecular portion.

26. The polyether NOP, method, composition, polymer, polyurethane, article, or foam of any other embodiment wherein the polyether NOP has an average of at least about any of 0.5, 0.8, or 1, optional at most about any of 8, 5 or 3 fatty acid moieties oligomerized on each active hydrogen atom of the polyether initiator.

27. The polyether NOP, method, composition, polymer, polyurethane, article, or foam of any other embodiment wherein the polyether NOP has sufficient reactivity to react with a selected isocyante or combination thereof such that a foam having desirable properties are is produced, advantageously, when the active hydrogen functional groups of the polyether NOP are all hydroxyl groups, an average of at least about 75, 76, 77, 78, 79, or 80 percent of the number of hydroxyl groups on the polyether NOP are primary hydroxyl groups.

28. The polyether NOP, method, composition, polymer, polyurethane, article, or foam of any other embodiment wherein at least one polyether NOP is used to make a polyurethane and the amount of polyether NOP or combined polyether NOP's is at least about any of 1, 20, or 50 and optionally at most about any of 100, 95, 90, or 80 weight percent of total polyols used with one or more non NOP making up the remainder.

29. The polyether NOP, method, composition, polymer, polyurethane, article, or foam of any other embodiment wherein at least one polyether NOP is used in combination with at least one non-polyether NOP (a NOP not having the natural oil moieties separated by a polyether of at least about 480 equivalent weight and at least about 19 ether units) in the substantial absence of a conventional polyether polyol to make a polyurethane and the amount of polyether NOP or combined polyether NOP's is at least about any 5, more preferably at least about 10 or 20 and optionally at most about any of 95, 90, 85 or 80 weight percent of total polyols used, with at least one NOP which is a conventional NOP making up the remainder.

30. The polyether NOP, method, composition, polymer, polyurethane, article, or foam of any other embodiment wherein at least one polyether NOP is used in combination with at least one non-polyether NOP and at least one conventional polyol, and the amount of polyether NOP or combined polyether NOP's is at least about any of 5, 10 or 20 and optionally at most about 95, 90, 85 or 80 weight percent of total polyols used; and independently, the conventional polyol or combination thereof is present in an amount of at least about 5, 10 or 20, and optionally at most about 95,90, 85 or 80 weight percent of total polyols used, with at least one NOP that is conventional NOP according to the practice of the invention making up the remainder.

31. The foam of any other embodiment having at least one and with increasing preference 2, 3, 4, 5 or 6 of: (a) greater miscibility or compatibility with conventional polyether polyols, (b) greater miscibility or compatibility with water, (c) greater processibility in making a polyurethane, preferably a polyurethane foam, (d) increased tensile strength in a polyurethane; (e) increased elongation in a polyurethane, or (f) increased resiliency in a polyurethane, where, in each instance, the comparative polyurethane is produced by the same method using the same formulation except that the polyether NOP of the invention is replaced by an operably comparable conventional NOP; preferably including at least one of (a) or (c) or, more preferably both (a) and (c). The operably comparable conventional NOP preferably has an equivalent weight within about 15, more preferably within about 10 weight percent of the polyether NOP; preferably has at least one of (a) an average functionality within about 1 unit, more preferably within about 0.7 units, of the polyether NOP; or (b) an average percentage of primary active hydrogen groups preferably within about 2 percentage points, more preferably within about 1 percentage points of that of the polyether NOP; most preferably both (a) and (b).

1. A polyether NOP comprising at least two natural oil moieties separated by a molecular structure having an average of at least about 19 ether groups between any 2 of the natural oil moieties or by a polyether molecular structure having an equivalent weight of at least about 480, said polyol referred to hereinafter as a polyether NOP.

2. The polyether NOP of claim 1 wherein the polyether NOP is produced using a natural oil or derivative thereof to react with a polyether initiator.

3. The polyether NOP of claim 1 wherein the polyether initiator has an average equivalent weight of at least about 600 and at most about 3000 per active hydrogen group.

4. The polyether NOP of claim 2 wherein the polyether initiator has an active hydrogen functionality of at least about 2 and at most about 8.

5. The polyether NOP of claim 1 wherein the polyether NOP is produced using a combination of polyether initiators.

6. The polyether NOP of claim 1 wherein the polyether initiators is depicted by
R((OCH₂CHY)_{b}—XH)ₚ, Formula 1:
where Y is a H CH₃ or higher alkyl group or mixture thereof; X is an active hydrogen group; p is 1 to 8; b is sufficient to result in an equivalent weight per active hydrogen group of at least about 480; the number of ether units in an arm of the polyether initiator, b, is preferably at least about 9.5; R represents a linear, cyclic chain or combination thereof of alkane (C—C), alkene (C═C), ether (C—O—C) linkage or combination thereof; R has from 1 to 30 carbon atoms.

7. The polyether NOP of claim 6 wherein the natural oil or derivative thereof comprises at least one vegetable oil based (VOB) monomer resulting from hydroformylation at sites of unsaturation on fatty acids derived from at least one natural oil.

8. The polyether NOP of claim 7 wherein the polyether NOP is represented by Formula 2: R(OCH₂CHY)_{b} XQ)ₚ, wherein R, X, b, and p are as defined for Formula 1 and each Q independently represents at least one natural oil moiety.

9. The polyether NOP of claim 8, wherein each polyether NOP has an average of at least about 0.5 to at most about 8 fatty acid moieties oligomerized on each active hydrogen atom of the polyether initiator.

10. The polyether NOP of claim 1 wherein the natural oil moiety or the natural oil or derivative thereof used to make the polyether NOP comprises fatty acids of which at least about 70 percent by weight are unsaturated fatty acids.

11. The polyether NOP of claim 1 having at least one characteristic of: (a) greater miscibility or compatibility with conventional polyether polyols, (b) having greater miscibility or compatibility with water, (c) offering greater processability in making a polyurethane foam (d) increased tensile strength in a polyurethane; (e) increased elongation in a polyurethane, or (f) increased resiliency in a polyurethane, where, in each instance, the comparative polyurethane is produced by the same method and using the same formulation except that the polyether NOP is replaced by an operably comparable conventional NOP, that is an NOP having the natural oil moieties separated by a group having an equivalent weight of less than 480 or not having ether groups and having an equivalent weight within about 15 percent of that of the polyether NOP.

12. The polyether NOP of claim 11 wherein the NOP has at least 2 of the listed characteristics.

13. A polymer comprising the reaction product of a polyether NOP of claim 1 and at least one other monomer different from a polyether NOP and reactive therewith.

14. The polymer of claim 13 which is a polyurethane wherein at least one other monomer is at least one polyisocyanate.

15. The polymer of claim 13 wherein the polyether NOP is used in combination with at least one other polyol and the polyether NOP or combination thereof is present in an amount of at least about 10 up to at most about 95 weight percent.

16. The polymer of claim 15 wherein the polyether NOP is used in combination with at least one polyether polyol.

17. An article comprising at least one polymer of claim 13.

18. The article of claim 17 wherein the polymer is at least partially in the form of a foam.

19. The article of claim 18 wherein the foam is a high resiliency foam, having a resiliency of at least about 40 percent.

20. A polyurethane foam comprising the reaction product of at least:
a polyether NOP, wherein the polyether NOP comprises at least two natural oil moieties separated by a molecular structure having an average of at least about 19 ether groups between any 2 of the natural oil moieties or by a polyether molecular structure having an equivalent weight of at least about 480, said polyol referred to hereinafter as a polyether NOP; and
an isocyanate, wherein polyurethane foam has a resiliency of at least about 55 percent.

21. The polyurethane foam of claim 20, wherein the resiliency is at least about 60 percent
